(19) United States
(12) Patent Application Publication
(10) Pub. No.: US 2011/0135729 A1
(43) Pub. Date: Jun. 9, 2011

(54) Methods for Controlling Heat Generation of Magnetic Nanoparticles and Heat Generating Nanomaterials

(75) Inventors: Jin Woo Cheon, Seoul (KR); Jung Tak Jang, Seoul (KR)

(73) Assignee: Industry-Academic Cooperation Foundation Yonsei University, Seoul (KR)

(21) Appl. No.: 12/993,329

(22) PCT Filed: May 20, 2009
(86) PCT No.: PCT/KR09/02662

§ 371 Date: Feb. 10, 2011

(30) Foreign Application Priority Data
May 20, 2008 (KR) 10-2008-0046591

(51) Int. Cl. A61K 33/30; A61K 9/10; A61K 33/32; A61P 43/00; A61P 35/00; B82Y 5/00

(52) U.S. Cl. 424/484; 424/641; 424/639; 977/915

(57) ABSTRACT

The present invention relates to a method for controlling heat generation of a magnetic nanomaterial, comprising the steps of: (a) mixing (i) a nanomaterial precursor comprising a metal precursor material and a predetermined amount of a zinc precursor with (ii) a reaction solvent; and (b) preparing a zinc-containing magnetic nanomaterial from the mixture of step (a) comprising a zinc doped metal oxide nanomaterial matrix; and wherein a specific loss power of the zinc-containing magnetic nanomaterial is varied depending an amount of zinc to be doped, whereby the heat generation of the magnetic nanomaterial is controlled. In addition, the present invention relates to a heat-generating nanoparticle and a composition for hyperthermia. The present invention suggests a novel approach to improve a heat generation of a magnetic nanomaterial. According to the present invention, the specific loss power can be controlled by changing a zinc-content to be introduced into nanomaterials and therefore a composition for hyperthermia showing controlled heat generation potential can be successfully provided.

### BACKGROUND OF THE INVENTION

1. Field of the Invention

The present invention relates to a method for controlling heat generation of magnetic nanomaterials, a heat-generating nanomaterial, and a composition for hyperthermia.

2. Description of the Related Art

Nanomaterial exhibits new physiochemical characteristics different from bulk material when its size is reduced to a nano-scale particle. The intensive researches for the nanomaterials permit nanomaterials to be precisely controlled in their composition and shape as well as the size, enabling that the physiochemical properties in a nano-region can be controlled like those in a bulk-region. Using these novel properties, the nanomaterials has been currently used in a variety of applications such as a catalyst of chemical reactions, preparation of next generation nano devices, development of new sources of energy, and cancer diagnosis and therapy through combinations with a biomedical science (nano-medicine).

Since the magnetic nanomaterials among nanomaterials has a unique magnetic property, it has wide applications including (i) cell separation; (ii) magnetic resonance imaging (MRI) to remarkably enhance the signal of non-invasive imaging; (iii) probe of magnetic tweezers which is able to observe physical features of intracellular portion or cell surface by imposing external influence inside or on a cell. In particular, various applications using heat generated from the magnetic nanomaterial have been vigorously studied.

Of them, magnetic nanomaterials having unique magnetic property generate heat under a magnetic field of high frequency by (a) Brownian relaxation caused by rotation of nanomaterials dispersed in a liquid solution and (b) Neel relaxation caused from energy barrier of internal spin of nanomaterials (E=KV, where K is the anisotropy constant and V is the volume of the nanomaterials) (J. Mater. Chem., 2004, 14, 2161-2175). Using heat generated thus, the magnetic nanomaterial may be applied to a multitude of; heat-generating devices or technologies. In medical area, heat generated from the magnetic material under a magnetic field of high frequency has been used in hyperthermia for various diseases and disorders such as cancer.

Heat generated by magnetic nanomaterials may be quantitated by specific loss power (SLP). As referred to R. E. Rosensweig (J. Magn. Magn. Mater. 2002, 252, 370-374.), the value of specific loss power was determined according to various factors of materials, in particular a spin anisotropy and a saturation magnetism (Mₛ).

In this context, various research teams have made intensive studies to develop nanomaterials having higher specific loss power. Up to date, the applicable fields of heat generation using nanomaterials are as follows:

U.S. Pat. No. 7,282,479 discloses a hyperthermia agent for malignant tumors comprising the magnetic fine particles such as ferrite, magnetite or permalloy.

US Pat. Appln. No. 2005-0090732 discloses a targeted thermotherapy using an iron oxide.

U.S. Pat. No. 6,541,039 discloses a hyperthermia method using an iron oxide coated by a silica or polymer.

WO2006/102307 discloses a method for hyperthermia using the magnetic nanomaterial in which a core coated with a noble metal is surrounded by other organic shell, followed by packing with an antibody or a fluorescent material.

However, the above-described studies suggested only that each magnetic nanomaterials has heat-generating effect. In addition, the researches to significantly enhance or effectively control heat generation ability of nanoparticles have been not emerged yet. Therefore, the development of the magnetic nanomaterials having controllable specific loss power according to enhanced specific loss power and utilization has been urgently demanded in the senses that the magnetic nanomaterials having controllable specific loss power is needed for effective disease treatment.

Throughout this application, various publications and patents are referred and citations are provided in parentheses. The disclosures of these publications and patents in their entities are hereby incorporated by references into this application in order to fully describe this invention and the state of the art to which this invention pertains.

### SUMMARY OF THE INVENTION

The present inventors have made intensive studies to develop a nanomaterial for overcoming a problem in which a conventional magnetic nanomaterial has low specific loss power under a AC magnetic field of high frequency. To accomplish this purpose, we have first made researches to suggest novel methods to control spin anisotropy and saturation magnetism (Mₛ). As results, we have found that spin anisotropy or saturation magnetism (Mₛ) of nanomaterials could be improved by controlling zinc-contents in magnetic nanomaterials, and consequently the specific loss power of nanomaterials could be controlled or enhanced.

Accordingly, it is an object of this invention to provide a method for controlling a specific loss power of a magnetic nanomaterial.

It is another object of this invention to provide a heat-generating composition comprising a zinc-containing nanomaterial.

It is still another object of this invention to provide a composition for hyperthermia.

Other objects and advantages of the present invention will become apparent from the following detailed description together with the appended claims and drawings.

### BRIEF DESCRIPTION OF THE DRAWINGS

FIG. 1 shows TEM (transmission electron microscopy) images of zinc-containing metal oxide nanomaterials. FIGS. 1a-1e and FIGS. 1f-1j represent manganese ferrite nanoparticles and iron oxide nanoparticles containing various zinc compositions (x=0.1, 0.2, 0.3, 0.4, and 0.8), respectively. All nanoparticles have an equivalent size of 15 nm and exhibit a homogeneous size distribution (δ<5%).

FIGS. 2a-2b represent EDAX (Energy dispersive X-ray spectroscopy) and ICP-AES (Inductively coupled plasma atomic emission spectroscopy) analysis according to various compositions of manganese ferrite nanoparticles (ZnₓMn₁₋ₓFe₂O₄; FIGS. 2a-(a) and (b)) and iron oxide nanoparticles (ZnₓFe₃₋ₓO₄; FIGS. 2b-(c) and (d)) containing zinc (x=0.1, 0.2, 0.3, 0.4, 0.8). EDAX and ICP-AES of each ZnₓMn₁₋ₓFe₂O₄ (FIGS. 2a-(a) and (b)) and ZnₓFe₃₋ₓO₄ (FIGS. 2b-(c) and (d)) are represented in FIGS. 2a-2b.

FIG. 3 is a graph measuring saturation magnetism according to ZnₓMn₁₋ₓFe₂O₄ and ZnₓFe₃₋ₓO₄ containing various zinc compositions (x=0, 0.1, 0.2, 0.3, 0.4, and 0.8). It is likely that saturation magnetism is enhanced depending on increase in zinc-content of from 0 to 0.4, but is reduced in zinc-content of 0.8.

FIG. 4 is a graph representing a time-dependent temperature change of the synthesized iron oxide nanoparticle in an alternative current magnetic field.

FIG. 5 represents histograms comparing specific loss powers of ZnₓMn₁₋ₓFe₂O₄ and ZnₓFe₃₋ₓO₄ according to various zinc compositions (x=0, 0.1, 0.2, 0.4, and 0.8), suggesting that heat generation of nanoparticles is controlled by zinc-contents. In particular, the specific loss power is highest in zinc-content (x=0.2).

FIG. 6 represents histograms comparing specific loss powers of various metal oxide (MFe₂O₄, M=Mn²⁺, Fe²⁺, Co²⁺, and Ni²⁺) containing an equivalent amount of zinc (x=0.2, Zn_{0.2}M_{0.8}Fe₂O₄, M=Mn²⁺, Fe²⁺, Co²⁺, and Ni²⁺), demonstrating that the specific loss power is increased depending on zinc addition.

FIG. 7 shows TEM and EDAX analysis of iron oxide nanoparticles (ZnₓFe₃₋ₓO₄, x=0.2, 0.4) with different zinc-contents synthesized in an aqueous solution.

FIG. 8 is (a) histogram comparing heat-generation coefficient of zinc-containing manganese ferrite nanoparticle (Zn_{0.4}Mn_{0.6}Fe₂O₄) with that of commercialized Feridex and (b) cell apoptosis assay compared between manganese ferrite nanoparticle (Zn_{0.4}Mn_{0.6}Fe₂O₄) and commercialized Feridex, and (c)-(d) microscopic images thereof. In FIG. 8a, it is demonstrated that Zn_{0.4}Mn_{0.6}Fe₂O₄ has the specific loss power 4-fold higher than Feridex. In FIG. 8b, it is evident that Zn_{0.4}Mn_{0.6}Fe₂O₄ has the cell viability about 6.5-fold higher than Feridex in the case the equal amount of nanoparticles is added to HeLa cells. In addition, FIGS. 8c-8d shows microscopic images observed after Zn_{0.4}Mn_{0.6}Fe₂O₄ and Feridex is treated into HeLa cells.

### DETAILED DESCRIPTION OF THE INVENTION

In one aspect of this invention, there is provided a method for controlling specific loss power of a magnetic nanomaterial, comprising the steps of: (a) mixing (i) a nanomaterial precursor comprising a metal precursor material and a predetermined amount of a zinc precursor with (ii) a reaction solvent; and (b) preparing a zinc-containing magnetic nanomaterial from the mixture of step (a) comprising a zinc doped metal oxide nanomaterial matrix; and wherein a specific loss power of the zinc-containing magnetic nanomaterial is varied depending an amount of zinc to be doped, whereby the heat generation of the magnetic nanomaterial is controlled.

The present inventors have found that a magnetic anisotropy of nanomaterials could be remarkably enhanced by introduction of zinc into various metal-containing metal oxide nanomaterials and thus heat-generating nanomaterials comprising zinc-containing metal oxide exhibit dramatically increased specific loss power.

The term “zinc-containing magnetic nanomaterial” refers to a nanomaterial in which a zinc atom is added to the metal oxide nanomaterial matrix to substitute a metal atom or to be added to a vacant interstitial hole. The term “matrix” means an inorganic core of nanoparticle and also a mother body enabling to add or subtract various elements. Interestingly, the zinc-containing metal oxide nanomaterials have an enhanced specific loss power compared to original zinc-free metal oxide nanomaterial matrix, and their specific loss power is able to be controlled depending on zinc-content.

The term “metal oxide nanomaterial matrix” refers to an inorganic nano-material of a mother body to which zinc is added. The metal oxide that is used as the matrix includes a nanoparticle having the following formula 1 or 2:

MₐO_{b} Formula 1

[0<a≦16, 0<b≦8; M represents a magnetic metal atom (preferably transition metal elements, Lanthanide metal elements or Actinide metal elements, more preferably transition metal elements selected from the group consisting of Ba, Mn, Co, Ni and Fe, or Lanthanide metal elements selected from the group consisting of Gd, Er, Ho, Dy, Tb, Sm and Nd, and most preferably Lanthanide metal elements selected from the group consisting of Mn, Co, Ni, Fe, Gd, Er, Ho, Dy, Tb, Sm and Nd) or the alloy thereof]; or

M_{c}M′_{d}Oₑ Formula 2

(0<c≦16, 0<d≦16, 0<e≦8;

M represents the magnetic metal atom or the alloy thereof; M′ represents an element selected from the group consisting of Group 1 metal elements, Group 2 metal elements, Group 12 elements, Group 13 elements, Group 14 elements, Group 15 elements, transition metal elements, Lanthanide metal elements and Actinide metal elements, and preferably an element selected from the group consisting of Li, Na, K, Rb, Cs, Be, Mg, Ca, Sr, Ba, Ra, Ge, Ga, In, Si, Bi, Ti, V, Cr, Mn, Fe, Co, Ni, Cu, Y, Zr, Nb, Mo, Tc, Ru, Rh, Pd, Ag, Cd, Hf, Ta, W, Re, Os, Ir, Pt, Au, Hg, Lanthanide metal elements and Actinide metal elements).

The above-described metal oxide in which zinc atoms are added to the metal oxide nanomaterial matrix to substitute a portion of metal atoms or to be added to a vacant interstitial hole includes a magnetic nanomaterial represented by the following formula 3 or 4:

Zn_{f}M_{a-f}O_{b} Formula 3

(0<f<8, 0<a≦16, 0<b≦8, 0<f/(a-f)<10; M represents the magnetic metal atom or the alloy thereof); or

Zn_{g}M_{c-g}M′_{d}Oₑ Formula 4

(0<g<8, 0<c≦16, 0<d≦16, 0<e≦8, 0<g/{(c-g)+d}<10; M represents the magnetic metal atom or the alloy thereof; M′ represents an element selected from the group consisting of Group 1 metal elements, Group 2 metal elements, Group 12 elements, Group 13 elements, Group 14 elements, Group 15 elements, transition metal elements, Lanthanide metal elements and Actinide metal elements, and preferably an element selected from the group consisting of Li, Na, K, Rb, Cs, Be, Mg, Ca, Sr, Ba, Ra, Ge, Ga, In, Si, Ge, Bi, Ti, V, Cr, Mn, Fe, Co, Ni, Cu, Y, Zr, Nb, Mo, Tc, Ru, Rh, Pd, Ag, Cd, Hf, Ta, W, Re, Os, Ir, Pt, Au, Hg, Lanthanide metal elements and Actinide metal elements).

According to a preferable embodiment, the metal oxide used as the matrix includes a nanoparticle represented by the following formula 5:

M″ₕFeᵢOⱼ Formula 5

(0<h≦16, 0<i≦8, 0<j≦8; M″ represents the magnetic metal atom or the alloy thereof).

Preferably, the zinc-containing magnetic nanomaterial comprising a zinc doped metal oxide nanomaterial matrix includes a nanoparticle represented by the following formula 6:

ZnₖM″ₕ₋ₖFeᵢOⱼ Formula 6

(0<k<8, 0<h≦16, 0<i≦8, 0<j≦8, 0<k/{(h-k)+i}<10; M″ represents the magnetic metal atom or the alloy thereof).

More preferably, the metal oxide used as the matrix includes a nanoparticle represented by the following formula 7 or 8:

FeₗOₘ (0<l≦18, 0<m≦8); or Formula 7

MnₙFeₒOₚ (0<n≦8, 0<o≦8, 0<p≦8). Formula 8

More preferably, the zinc-containing magnetic nanomaterial comprising a zinc doped metal oxide nanomaterial matrix includes a nanoparticle represented by the following formula 9 or 10:

Zn_{g}Fe_{l-q}Oₘ (0<q<8, 0<l≦18, 0<m≦8, 0<q/(l-q)<10); or Formula 9

ZnᵣMnₙ₋ᵣFeₒOₚ (0<r<8, 0<n≦8, 0<o≦8, 0<p≦8, 0<r/{(n-r)+o}<10). Formula 10

The term “zinc-containing” means that a zinc atom is put into a tetrahedron hole or an octahedron hole among cationic interstitial holes that are present among oxygen atoms in a crystalline structure of the metal oxide nanomaterial matrixes. Thus, when the zinc-containing water-soluble or water-dispersible metal oxide nanomaterials are grown, after the metal oxide nanomaterial matrixes are first synthesized, the metal atoms that are present in the tetrahedron hole or octahedron hole on the matrix are substituted with zinc, or when the metal oxide nanomaterial matrixes are growing, the metal atom and zinc are simultaneously introduced to synthesize the nanoparticles so that zinc is put into the tetrahedron hole or the octahedron hole among the cation interstitial holes of the oxygen atoms (example: ZnO+Fe₂O₃→ZnFe₂O₄). In particular, zinc in tetrahedron hole is important because it plays a role to enhance the magnetic moment of nanoparticles. The composition of metal oxide of nanoparticle where zinc is to be added may be non-stoichiometric.

In the zinc-containing magnetic nanomaterial of the present invention, a stoichiometric content ratio of zinc and other metal materials is as follows: 0.001<‘zinc/(entire metal component-zinc)’<10, more preferably 0.01<‘zinc/(entire metal component-zinc)’<1, and most preferably 0.03<‘zinc/(entire metal component-zinc)’<0.5. When zinc is contained as the above, high saturation magnetism can be obtained.

According to this invention, heat generation of nanoparticles may be controlled by changing zinc-content contained in the nanoparticles.

To obtain zinc-containing metal oxide nanomaterial for heat generation, the nanoparticles which are synthesized by crystal growth in an aqueous solution or an organic solvent of nanoparticle precursors through a chemical reaction, or are synthesized in an organic solvent may be solubilized in water using a phase transfer method. The zinc-containing metal oxide magnetic nanomaterial according to the present invention is not limited depending on the formulations and has enhanced heat-generating effects.

As an illustrative example among the preferable methods for preparing the present nanoparticles, the nanoparticles may be prepared through the following steps: (i) preparing a mixture solution in which a nanoparticle precursor containing a magnetic and/or a metal precursor material is added to a surfactant or an organic solvent including a surfactant, (ii) fabricating the magnetic or metal oxide nanomaterial whereby the precursors is decomposed by heating the mixture solution at high temperature (e.g., 150-500° C.), and (iii) separating the nanoparticles.

As the nanoparticle precursors, a metal nitrate-based compound, a metal sulfate-based compound, a metal acetylacetonate-based compound, a metal fluoroacetoacetate-based compound, a metal halide-based compound, a metal perchlorate-based compound, a metal alkyloxide-based compound, a metal sulfamate-based compound, a metal stearate-based compound or an organometallic compound may be used, but not limited to.

As the organic solvents, a benzene-based solvent, a hydrocarbon solvent, an ether-based solvent, a polymer solvent or an ionic liquid solvent may be used, and preferably benzene, toluene, halobenzene, octane, nonane, decane, benzyl ether, phenyl ether, hydrocarbon ether, a polymer solvent or an ionic liquid solvent may be used, but not limited to.

In addition, the nanoparticles synthesized according to the above-described manufacturing method may be used in aqueous solution by phase transfer method using a water-soluble multi-functional ligand.

The term “water-soluble multi-functional ligand” refers to a ligand that may be bound to zinc-containing metal oxide nanomaterial, to solubilize in water and stabilize the nanoparticles, and may allow the nanoparticles to be bound by biologically/chemically active materials.

The water-soluble multi-functional ligand can include (a) an adhesive region (L_{I}), and further can include (b) a reactive region (L_{II}), (c) a cross-linking region (L_{III}), or (d) a reactive & cross-linking region (L_{II}-L_{III}) which includes both the active ingredient-binding region (L_{II}) and the cross-linking region (L_{III}). Hereinafter, the water-soluble multi-functional ligand will be described in detail.

The term “adhesive region (L_{I})” refers to a portion of a multi-functional ligand including a functional group capable of binding to the nanoparticles, and preferably, to an end portion of the functional group. Accordingly, it is preferable that the adhesive region including the functional group should have high affinity with the materials constituting the nanoparticles. The nanoparticle can be attached to the adhesive region by an ionic bond, a covalent bond, a hydrogen bond, a hydrophobic interaction or a metal-ligand coordination bond. The adhesive region of the multi-functional ligand may be varied depending on the substances constituting the nanoparticles. For example, the adhesive region (L₁) using ionic bond, covalent bond, hydrogen bond or metal-ligand coordination bond may include —COOH, —NH₂, —SH, —CONH₂, —PO₃H, —OPO₃H₂, —SO₃H, —OSO₃H, —N₃, —NR₃OH(R═CₙH₂ₙ₊₁, 0≦n≦16), —OH, —SS—, —NO₂, —CHO, —COX (X═F, Cl, Br or I), —COOCO—, —CONN— or —CN, and the adhesive region (L_{I}) using hydrophobic interaction may include a hydrocarbon chain having two or more carbon atoms, but not limited to.

The term “reactive region (L_{II})” means a portion of the multi-functional ligand containing a functional group capable of binding to chemically or biologically active materials, and preferably the other end portion located at the opposite side from the adhesive region. The functional group of the reactive region may be varied depending on the type of active ingredient and their chemical formulae (Table 1). In this invention, the reactive region includes, but not limited to, —SH, —COOH, —CHO, —NH₂, —OH, —PO₃H, —OPO₃H₂, —SO₃H, —OSO₃H, —NR₃⁺X⁻ (R═CₙHₘ, 0≦n≦16, 0≦m≦34, X═OH, Cl or Br), NR₄⁺X⁻ (R═CₙHₘ, 0≦n≦16, 0≦m≦34, X═OH, Cl or Br), —N₃, —SCOCH₃, —SCN, —NCS, —NCO, —CN, —F, —Cl, —Br, —I, an epoxide group, a sulfonate group, a nitrate group, a phosphonate group, an aldehyde group, a hydrazone group, —C═C— and —C═C—.

The term “cross-linking region (L_{III})” refers to a portion of the multi-functional ligand including a functional group capable of cross-linking to an adjacent multi-functional ligand, and preferably a side chain attached to a central portion. The term “cross-linking” means that the multi-functional ligand is bound to another adjacent multi-functional ligand by intermolecular interaction. The intermolecular interaction includes, but not limited to, a hydrophobic interaction, a hydrogen bond, a covalent bond (e.g., a disulfide bond), a Van der Waals force and an ionic bond. Therefore, the cross-linkable functional group may be varioulsly selected according to the kind of the intermolecular interaction. For example, the cross-linking region may include —SH, —COOH, —CHO, —NH₂, —OH, —PO₃H, —OPO₃H₂, —SO₃H, —OSO₃H, Si—OH, Si(MeO)₃, —NR₃⁺X⁻(R═CₙHₘ, 0≦n≦16, 0≦m≦34, X═OH, Cl or Br), NR₄⁺X⁻ (R═CₙHₘ, 0≦n≦16, 0≦m≦34, X═OH, Cl or Br), —N₃, —SCOCH₃, —SCN, —NCS, —NCO, —CN, —F, —Cl, —Br, —I, an epoxy group, —ONO₂, —PO(OH)₂, —C═NNH₂, —C═C— and —C≡C— as the functional ligand, but not limited to.

TABLE 1
Exemplary functional groups of reactive region in multi-functional
ligand

| I | II | III |
|---|---|---|
| R—NH₂ | R′—COOH | R—NHCO—R′ |
| R—SH | R′—SH | R—SS—R′ |
| R—OH | R′-(Epoxy group) | R—OCH₂CH(OH)—R′ |
| R—NH₂ | R′-(Epoxy group) | R—NHCH₂CH(OH)—R′ |
| R—SH | R′-(Epoxy group) | R—SCH₂CH(OH)—R′ |
| R—NH₂ | R′—COH | R—N═CH—R′ |
| R—NH₂ | R′—NCO | R—NHCONH—R′ |
| R—NH₂ | R′—NCS | R—NHCSNH—R′ |
| R—SH | R′—COCH₃ | R—COCH₂S—R′ |
| R—SH | R′—O(C═O)X | R—S(C═O)O—R′ |
| R- | R′—SH | R—CH₂CH(NH₂)CH₂S—R′ |
| (Aziridine group) | | |
| R—CH═CH₂ | R′—SH | R—CH₂CH₂S—R′ |
| R—OH | R′—NCO | R—NHCOO—R′ |
| R—SH | R′—COCH₂X | R—SCH₂CO—R′ |
| R—NH₂ | R′—CON₃ | R—NHCO—R′ |
| R—COOH | R′—COOH | R—(C═O)O(C═O—R′) + |
| | | H₂O |
| R—SH | R′—X | R—S—R′ |
| R—NH₂ | R′CH₂C(NH²⁺)OCH₃ | R—NHC(NH²⁺)CH₂—R′ |
| R—OP(O²⁻)OH | R′—NH₂ | R—OP(O²⁻)—NH—R′ |
| R—CONHNH₂ | R′—COH | R—CONHN═CH—R′ |
| R—NH₂ | R′—SH | R—NHCO(CH₂)₂SS—R′ |

(I: functional group of reactive region in multi-functional ligand, II: active ingredient, III: exemplary bonds by reaction of I and II)

In this invention, the compound which originally contains the above-described functional group may be used as a water-soluble multi-functional ligand, but a compound modified or prepared so as to have the above-described functional group by a chemical reaction known in the art may be also used as a water-soluble multi-functional ligand.

The preferable multi-functional ligand of the present invention includes a monomer, a polymer, a carbohydrate, a protein, a peptide, a nucleic acid, a lipid and an amphiphilic ligand.

For the water-soluble nanoparticle of the present invention, one example of the preferable multi-functional ligand is a monomer which contains the functional group described above, and preferably dimercaptosuccinic acid, since it originally contains the adhesive region, the cross-linking region and the reactive region. That is, —COOH on one side of dimercaptosuccinic acid is bound to the magnetic signal generating core, and —COOH and —SH on the other end portion functions to bind to an active ingredient. In addition, —SH of dimercaptosuccinic acid acts as the cross-linking region by disulfide bond with another —SH. In addition to the dimercaptosuccinic acid, other compounds having —COOH as the functional group of the adhesive region and —COOH, —NH₂ or —SH as the functional group of the reactive region may be utilized as the preferable multi-functional ligand, but not limited to.

Still another example of the preferable water-soluble multi-functional ligand according to the present invention includes, but not limited to, one or more polymer selected from the group consisting of polyphosphagen, polylactide, polylactide-co-glycolide, polycaprolactone, polyanhydride, polymaleic acid, a derivative of polymaleic acid, polyalkylcyanoacrylate, polyhydroxybutylate, polycarbonate, polyorthoester, polyethylene glycol, poly-L-lysine, polyglycolide, polymethyl methacrylate and polyvinylpyrrolidone.

In the zinc-containing water-soluble metal oxide nanomaterial according to the present invention, preferable other examples of the multi-functional ligand are a peptide. Peptide is oligomer/polymer consisting of several amino acids. Since the amino acids have —COOH and —NH₂ functional groups in both ends thereof, peptides naturally have the adhesive region and the reactive region. In addition, the peptide that contains one or more amino acids having one or more of —SH, —COOH, —NH₂ and —OH as the side chain may be utilized as the preferable water-soluble multi-functional ligand.

In the water-soluble nanoparticles according to the present invention, still another example of the preferable multi-functional ligand is a protein. Protein is a polymer composed of more amino acids than peptides, that is, composed of several hundreds to several hundred thousands of amino acids. Proteins contains —COOH and —NH₂ functional group at both ends, and also contains a lot of functional groups such as —COOH, —NH₂, —SH, —OH, —CONH₂, and so on. Proteins may be used as the water-soluble multi-functional ligand because they naturally contain the adhesive region, the cross-linking region and the reactive region according to its structure as the above-described peptide. The representative examples of proteins which are preferable as the water-soluble multi-functional ligand include a structural protein, a storage protein, a transport protein, a hormone protein, a receptor protein, a contraction protein, a defense protein and an enzyme protein, and more specifically, albumin, antibody, antigen, avidin, cytochrome, casein, myosin, glycinin, carotene, collagen, globular protein, light protein, streptavidin, protein A, protein G, protein S, lectin, selectin, angiopoietin, anti-cancer protein, antibiotic protein, hormone antagonist protein, interleukin, interferon, growth factor protein, tumor necrosis factor protein, endotoxin protein, lymphotoxin protein, tissue plasminogen activator, urokinase, streptokinase, protease inhibitor, alkyl phosphocholine, surfactant, cardiovascular pharmaceutical protein, neuro pharmaceuticals protein and gastrointestinal pharmaceuticals, but not limited to.

Still another example of the preferable water-soluble multi-functional ligand in the present invention is a nucleic acid. The nucleic acid is oligomer consisting of many nucleotides. Since the nucleic acids have —PO₄⁻ and —OH functional groups in their both ends, they naturally have the adhesive region and the reactive region or the adhesive region and the cross-linking region (L_{I}-L_{II}). Therefore, the nucleic acids may be useful as the water-soluble multi-functional ligand in this invention. In some cases, the nucleic acid is preferably modified to have the functional group such as —SH, —NH₂, —COON or —OH at 3′- or 5′-terminal ends.

For the water-soluble nanoparticles according to the present invention, still another example of the preferable multi-functional ligand is an amphiphilic ligand including both a hydrophobic and a hydrophilic region. In the nanoparticles synthesized in an organic solvent, hydrophobic ligands having long carbon chains coat the surface. When amphilphilic ligands are added to the nanoparticle solution, the hydrophobic region of the amphiphilic ligand and the hydrophobic ligand on the nanoparticles are bound to each other through intermolecular interaction to stabilize the nanoparticles. Further, the outermost part of the nanoparticles shows the hydrophilic functional group, and consequently water-soluble nanoparticles can be prepared. The intermolecular interaction includes a hydrophobic interaction, a hydrogen bond, a Van der Waals force, and so forth. The portion which binds to the nanoparticles by the hydrophobic interaction is an adhesive region (L_{I}), and further the reactive region (L_{II}) and the cross-linking region (L_{I}) can be introduced therewith by an organo-chemical method. In addition, in order to increase the stability in an aqueous solution, amphiphilic polymer ligands with multiple hydrophobic and hydrophilic regions can be used. Cross-linking between the amphiphilic ligands can be also performed by a linker for enhancement of stability in an aqueous solution. As the phase transfer ligands, hydrophobic region of the amphiphilic ligand can be a linear or branched structure composed of chains containing two or more carbon atoms, more preferably an alkyl functional group such as ethyl, n-propyl, isopropyl, n-butyl, isobutyl, t-butyl, octyl, decyl, tetradecyl, hexadecyl, icosyl, tetracosyl, dodecyl, cyclopentyl or cyclohexyl; a functional group having an unsaturated carbon chain containing a carbon-carbon double bond, such as ethenyl, propenyl, isopropenyl, butenyl, isobutenyl, octenyl, decenyl or oleyl; or a functional group having an unsaturated carbon chain containing a carbon-carbon triple bond, such as propynyl, isopropynyl, butynyl, isobutynyl, octynyl or decynyl. In addition, examples of the hydrophilic region include a functional group being neutral at a specific pH, but being positively or negatively charged at a higher or lower pH such as —SH, —COON, —NH₂, —OH, —PO₃H, —PO₄H₂, —SO₃H, —SO₄H or —NR₄⁺X⁻. Furthermore, preferable examples thereof include a polymer and a block copolymer, wherein monomers used therefor include ethylglycol, acrylic acid, alkylacrylic acid, ataconic acid, maleic acid, fumaric acid, acrylamidomethylpropane sulfonic acid, vinylsulfonic acid, vinylphosphoric acid, vinyl lactic acid, styrenesulfonic acid, allylammonium, acrylonitrile, N-vinylpyrrolidone and N-vinylformamide, but not limited to.

Another example of the preferable water-soluble multi-functional ligand in the nanoparticle of the present invention is a carbohydrate. More preferably, the carbohydrate includes, but not limited to, glucose, mannose, fucose, N-acetyl glucomine, N-acetyl galactosamine, N-acetylneuraminic acid, fructose, xylose, sorbitol, sucrose, maltose, glycoaldehyde, dihydroxyacetone, erythrose, erythrulose, arabinose, xylulose, lactose, trehalose, mellibose, cellobiose, raffinose, melezitose, maltoriose, starchyose, estrodose, xylan, araban, hexosan, fructan, galactan, mannan, agaropectin, alginic acid, carrageenan, hemicelluloses, hypromellose, amylose, deoxyacetone, glyceraldehyde, chitin, agarose, dextrin, ribose, ribulose, galactose, carboxy methylcellulose, glycogen dextran, carbodextran, polysaccharide, cyclodextran, pullulan, cellulose, starch and glycogen.

In addition, the water-soluble zinc-containing metal oxide may be synthesized according to a chemical reaction in an aqueous solution. This method is to synthesize the zinc-containing water-soluble metal oxide nanomaterials by adding zinc ion precursor materials to the reaction solution containing the water-soluble multi-functional ligand. It may be performed according to a synthesis method (e.g., a coprecipitation method, a sol-gel method, a micelle method) of a conventional water-soluble nanoparticle known to those skilled in the art.

As the nanoparticle precursors, a metal nitrate-based compound, a metal sulfate-based compound, a metal acetylacetonate-based compound, a metal fluoroacetoaeetate-based compound, a metal halide-based compound, a metal perchlorate-based compound, a metal alkyloxide-based compound, a metal sulfamate-based compound, a metal stearate-based compound, a metal alkoxide-based compound or an organometallic compound may be used, but not limited to.

As the organic solvents, a benzene-based solvent, a hydrocarbon solvent, an ether-based solvent, a polymer solvent, an ionic liquid solvent, an alcohol-based solvent, a sulfoxide-based solvent or water may be used, and preferably benzene, toluene, halobenzene, octane, nonane, decane, benzyl ether, phenyl ether, hydrocarbon ether, a polymer solvent, diethylene glycol (DEG), water or an ionic liquid solvent may be used, but not limited to.

The zinc-containing metal oxide nanomaterials according to the above-described method have a uniform size distribution (δ<10%) and a high crystallinity. In addition, with the method, zinc-content in the nanoparticle matrix may be precisely controlled. In other words, by changing the ratio of zinc to other metal precursor material, the zinc-content in the nanoparticle can be controlled between 0.001<‘zinc/(entire metal component-zinc)’<10 in a stoichiometric ratio.

The hydrodynamic diameter of the final nanoparticles prepared by the present invention is in a range of preferably 1-1000 nm, more preferably 1-800 nm and most preferably 2-500 nm.

The illustrative examples of the zinc-containing magnetic nanomaterial synthesized according to the present method are as the following Table 2:

TABLE 2

| | | Zinc-content | | Core | Water-soluble | Saturation | |
|---|---|---|---|---|---|---|---|
| | | (=zinc/total | Chemical | size | multi-functional | magnetism | specific loss |
| No. | Matrix | metal − zinc) | formula | (nm) | ligand* | (emu/g) | power (W/g) |
| 1 | iron oxide | 0.154 | Zn_{0.4}Fe_{2.6}O₄ | 6 | DMSA | 92 | 496 |
| 2 | iron oxide | 0.154 | Zn_{0.4}Fe_{2.6}O₄ | 6 | TMAOH | 92 | 496 |
| 3 | iron oxide | 0.154 | Zn_{0.4}Fe_{2.6}O₄ | 6 | BSA | 92 | 496 |
| 4 | iron oxide | 0.154 | Zn_{0.4}Fe_{2.6}O₄ | 6 | carbodextran | 92 | 496 |
| 5 | iron oxide | 0.154 | Zn_{0.4}Fe_{2.6}O₄ | 9 | DMSA | 108 | 496 |
| 6 | iron oxide | 0.154 | Zn_{0.4}Fe_{2.6}O₄ | 9 | TMAOH | 108 | 496 |
| 7 | iron oxide | 0.154 | Zn_{0.4}Fe_{2.6}O₄ | 9 | BSA | 108 | 496 |
| 8 | iron oxide | 0.154 | Zn_{0.4}Fe_{2.6}O₄ | 9 | carbodextran | 108 | 496 |
| 9 | iron oxide | 0.154 | Zn_{0.4}Fe_{2.6}O₄ | 12 | DMSA | 136 | 496 |
| 10 | iron oxide | 0.154 | Zn_{0.4}Fe_{2.6}O₄ | 12 | TMAOH | 136 | 496 |
| 11 | iron oxide | 0.154 | Zn_{0.4}Fe_{2.6}O₄ | 12 | BSA | 136 | 496 |
| 12 | iron oxide | 0.154 | Zn_{0.4}Fe_{2.6}O₄ | 12 | carbodextran | 136 | 496 |
| 13 | iron oxide | 0.034 | Zn_{0.1}Fe_{2.9}O₄ | 15 | DMSA | 126 | 540 |
| 14 | iron oxide | 0.034 | Zn_{0.1}Fe_{2.9}O₄ | 15 | TMAOH | 126 | 540 |
| 15 | iron oxide | 0.034 | Zn_{0.1}Fe_{2.9}O₄ | 15 | BSA | 126 | 540 |
| 16 | iron oxide | 0.034 | Zn_{0.1}Fe_{2.9}O₄ | 15 | carbodextran | 126 | 540 |
| 17 | iron oxide | 0.071 | Zn_{0.2}Fe_{2.8}O₄ | 15 | DMSA | 138 | 694 |
| 18 | iron oxide | 0.071 | Zn_{0.2}Fe_{2.8}O₄ | 15 | TMAOH | 138 | 694 |
| 19 | iron oxide | 0.071 | Zn_{0.2}Fe_{2.8}O₄ | 15 | BSA | 138 | 694 |
| 20 | iron oxide | 0.071 | Zn_{0.2}Fe_{2.8}O₄ | 15 | carbodextran | 138 | 694 |
| 21 | iron oxide | 0.111 | Zn_{0.3}Fe_{2.7}O₄ | 15 | DMSA | 152 | — |
| 22 | iron oxide | 0.111 | Zn_{0.3}Fe_{2.7}O₄ | 15 | TMAOH | 152 | — |
| 23 | iron oxide | 0.111 | Zn_{0.3}Fe_{2.7}O₄ | 15 | BSA | 152 | — |
| 24 | iron oxide | 0.111 | Zn_{0.3}Fe_{2.7}O₄ | 15 | carbodextran | 152 | — |
| 25 | iron oxide | 0.154 | Zn_{0.4}Fe_{2.6}O₄ | 15 | DMSA | 161 | 496 |
| 26 | iron oxide | 0.154 | Zn_{0.4}Fe_{2.6}O₄ | 15 | TMAOH | 161 | 496 |
| 27 | iron oxide | 0.154 | Zn_{0.4}Fe_{2.6}O₄ | 15 | BSA | 161 | 496 |
| 28 | iron oxide | 0.154 | Zn_{0.4}Fe_{2.6}O₄ | 15 | carbodextran | 161 | 496 |
| 29 | iron oxide | 0.154 | Zn_{0.4}Fe_{2.6}O₄ | 15 | hypromelose | 161 | 496 |
| 30 | iron oxide | 0.154 | Zn_{0.4}Fe_{2.6}O₄ | 15 | neutrovidin | 161 | 496 |
| 31 | iron oxide | 0.154 | Zn_{0.4}Fe_{2.6}O₄ | 15 | antibody (IgG) | 161 | 496 |
| 32 | iron oxide | 0.364 | Zn_{0.8}Fe_{2.2}O₄ | 15 | DMSA | 115 | 458 |
| 33 | iron oxide | 0.364 | Zn_{0.8}Fe_{2.2}O₄ | 15 | TMAOH | 115 | 458 |
| 34 | manganese | 0.154 | Zn_{0.4}Mn_{0.6}Fe₂O₄ | 6 | DMSA | 107 | 472 |
| | ferrite | | | | | | |
| 35 | manganese | 0.154 | Zn_{0.4}Mn_{0.6}Fe₂O₄ | 6 | TMAOH | 107 | 472 |
| | ferrite | | | | | | |
| 36 | manganese | 0.154 | Zn_{0.4}Mn_{0.6}Fe₂O₄ | 6 | BSA | 107 | 472 |
| | ferrite | | | | | | |
| 37 | manganese | 0.154 | Zn_{0.4}Mn_{0.6}Fe₂O₄ | 6 | carbodextran | 107 | 472 |
| | ferrite | | | | | | |
| 38 | manganese | 0.154 | Zn_{0.4}Mn_{0.6}Fe₂O₄ | 9 | DMSA | 129 | 472 |
| | ferrite | | | | | | |
| 39 | manganese | 0.154 | Zn_{0.4}Mn_{0.6}Fe₂O₄ | 9 | TMAOH | 129 | 472 |
| | ferrite | | | | | | |
| 40 | manganese | 0.154 | Zn_{0.4}Mn_{0.6}Fe₂O₄ | 9 | BSA | 129 | 472 |
| | ferrite | | | | | | |
| 41 | manganese | 0.154 | Zn_{0.4}Mn_{0.6}Fe₂O₄ | 9 | carbodextran | 129 | 472 |
| | ferrite | | | | | | |
| 42 | manganese | 0.071 | Zn_{0.2}Mn_{0.8}Fe₂O₄ | 12 | DMSA | 135 | 667 |
| | ferrite | | | | | | |
| 43 | manganese | 0.071 | Zn_{0.2}Mn_{0.8}Fe₂O₄ | 12 | TMAOH | 135 | 667 |
| | ferrite | | | | | | |
| 44 | manganese | 0.071 | Zn_{0.2}Mn_{0.8}Fe₂O₄ | 12 | BSA | 135 | 667 |
| | ferrite | | | | | | |
| 45 | manganese | 0.071 | Zn_{0.2}Mn_{0.8}Fe₂O₄ | 12 | carbodextran | 135 | 667 |
| | ferrite | | | | | | |
| 46 | manganese | 0.111 | Zn_{0.3}Mn_{0.7}Fe₂O₄ | 12 | DMSA | 146 | — |
| | ferrite | | | | | | |
| 47 | manganese | 0.111 | Zn_{0.3}Mn_{0.7}Fe₂O₄ | 12 | TMAOH | 146 | — |
| | ferrite | | | | | | |
| 48 | manganese | 0.111 | Zn_{0.3}Mn_{0.7}Fe₂O₄ | 12 | BSA | 146 | — |
| | ferrite | | | | | | |
| 49 | manganese | 0.111 | Zn_{0.3}Mn_{0.7}Fe₂O₄ | 12 | carbodextran | 146 | — |
| | ferrite | | | | | | |
| 50 | manganese | 0.154 | Zn_{0.4}Mn_{0.6}Fe₂O₄ | 12 | DMSA | 153 | 472 |
| | ferrite | | | | | | |
| 51 | manganese | 0.154 | Zn_{0.4}Mn_{0.6}Fe₂O₄ | 12 | TMAOH | 153 | 472 |
| | ferrite | | | | | | |
| 52 | manganese | 0.154 | Zn_{0.4}Mn_{0.6}Fe₂O₄ | 12 | BSA | 153 | 472 |
| | ferrite | | | | | | |
| 53 | manganese | 0.154 | Zn_{0.4}Mn_{0.6}Fe₂O₄ | 12 | carbodextran | 153 | 472 |
| | ferrite | | | | | | |
| 54 | manganese | 0.154 | Zn_{0.4}Mn_{0.6}Fe₂O₄ | 12 | hypromelose | 153 | 472 |
| | ferrite | | | | | | |
| 55 | manganese | 0.154 | Zn_{0.4}Mn_{0.6}Fe₂O₄ | 12 | neutrovidin | 153 | 472 |
| | ferrite | | | | | | |
| 56 | manganese | 0.154 | Zn_{0.4}Mn_{0.6}Fe₂O₄ | 12 | antibody (IgG) | 153 | 472 |
| | ferrite | | | | | | |
| 57 | manganese | 0.034 | Zn_{0.1}Mn_{0.9}Fe₂O₄ | 15 | DMSA | 140 | 451 |
| | ferrite | | | | | | |
| 58 | manganese | 0.034 | Zn_{0.1}Mn_{0.9}Fe₂O₄ | 15 | TMAOH | 140 | 451 |
| | ferrite | | | | | | |
| 59 | manganese | 0.034 | Zn_{0.1}Mn_{0.9}Fe₂O₄ | 15 | BSA | 140 | 451 |
| | ferrite | | | | | | |
| 60 | manganese | 0.034 | Zn_{0.1}Mn_{0.9}Fe₂O₄ | 15 | carbodextran | 140 | 451 |
| | ferrite | | | | | | |
| 61 | manganese | 0.071 | Zn_{0.2}Mn_{0.8}Fe₂O₄ | 15 | DMSA | 154 | 667 |
| | ferrite | | | | | | |
| 62 | manganese | 0.071 | Zn_{0.2}Mn_{0.8}Fe₂O₄ | 15 | TMAOH | 154 | 667 |
| | ferrite | | | | | | |
| 63 | manganese | 0.071 | Zn_{0.2}Mn_{0.8}Fe₂O₄ | 15 | BSA | 154 | 667 |
| | ferrite | | | | | | |
| 64 | manganese | 0.071 | Zn_{0.2}Mn_{0.8}Fe₂O₄ | 15 | carbodextran | 154 | 667 |
| | ferrite | | | | | | |
| 65 | manganese | 0.111 | Zn_{0.3}Mn_{0.7}Fe₂O₄ | 15 | DMSA | 166 | — |
| | ferrite | | | | | | |
| 66 | manganese | 0.111 | Zn_{0.3}Mn_{0.7}Fe₂O₄ | 15 | TMAOH | 166 | — |
| | ferrite | | | | | | |
| 67 | manganese | 0.111 | Zn_{0.3}Mn_{0.7}Fe₂O₄ | 15 | BSA | 166 | — |
| | ferrite | | | | | | |
| 68 | manganese | 0.111 | Zn_{0.3}Mn_{0.7}Fe₂O₄ | 15 | carbodextran | 166 | — |
| | ferrite | | | | | | |
| 69 | manganese | 0.154 | Zn_{0.4}Mn_{0.6}Fe₂O₄ | 15 | DMSA | 175 | 472 |
| | ferrite | | | | | | |
| 70 | manganese | 0.154 | Zn_{0.4}Mn_{0.6}Fe₂O₄ | 15 | TMAOH | 175 | 472 |
| | ferrite | | | | | | |
| 71 | manganese | 0.154 | Zn_{0.4}Mn_{0.6}Fe₂O₄ | 15 | BSA | 175 | 472 |
| | ferrite | | | | | | |
| 72 | manganese | 0.154 | Zn_{0.4}Mn_{0.6}Fe₂O₄ | 15 | carbodextran | 175 | 472 |
| | ferrite | | | | | | |
| 73 | manganese | 0.154 | Zn_{0.4}Mn_{0.6}Fe₂O₄ | 15 | hypromelose | 175 | 472 |
| | ferrite | | | | | | |
| 74 | manganese | 0.154 | Zn_{0.4}Mn_{0.6}Fe₂O₄ | 15 | neutrovidin | 175 | 472 |
| | ferrite | | | | | | |
| 75 | manganese | 0.154 | Zn_{0.4}Mn_{0.6}Fe₂O₄ | 15 | antibody (IgG) | 175 | 472 |
| | ferrite | | | | | | |
| 76 | manganese | 0.364 | Zn_{0.8}Mn_{0.2}Fe₂O₄ | 15 | DMSA | 138 | 379 |
| | ferrite | | | | | | |
| 77 | manganese | 0.364 | Zn_{0.8}Mn_{0.2}Fe₂O₄ | 15 | TMAOH | 138 | 379 |
| | ferrite | | | | | | |
| 78 | cobalt ferrite | 0.111 | Zn_{0.3}Co_{0.7}Fe₂O₄ | 12 | DMSA | | — |
| 79 | cobalt ferrite | 0.111 | Zn_{0.3}Co_{0.7}Fe₂O₄ | 12 | TMAOH | | — |
| 80 | cobalt ferrite | 0.111 | Zn_{0.3}Co_{0.7}Fe₂O₄ | 12 | BSA | | — |
| 81 | cobalt ferrite | 0.111 | Zn_{0.3}Co_{0.7}Fe₂O₄ | 12 | carbodextran | | — |
| 82 | cobalt ferrite | 0.154 | Zn_{0.4}Co_{0.6}Fe₂O₄ | 12 | DMSA | | 433 |
| 83 | cobalt ferrite | 0.154 | Zn_{0.4}Co_{0.6}Fe₂O₄ | 12 | TMAOH | | 433 |
| 84 | cobalt ferrite | 0.154 | Zn_{0.4}Co_{0.6}Fe₂O₄ | 12 | BSA | | 433 |
| 85 | cobalt ferrite | 0.154 | Zn_{0.4}Co_{0.6}Fe₂O₄ | 12 | carbodextran | | 433 |
| 86 | nickel ferrite | 0.111 | Zn_{0.3}Ni_{0.7}Fe₂O₄ | 12 | DMSA | | — |
| 87 | nickel ferrite | 0.111 | Zn_{0.3}Ni_{0.7}Fe₂O₄ | 12 | TMAOH | | — |
| 88 | nickel ferrite | 0.111 | Zn_{0.3}Ni_{0.7}Fe₂O₄ | 12 | BSA | | — |
| 89 | nickel ferrite | 0.111 | Zn_{0.3}Ni_{0.7}Fe₂O₄ | 12 | carbodextran | | — |
| 90 | nickel ferrite | 0.154 | Zn_{0.4}Ni_{0.6}Fe₂O₄ | 12 | DMSA | | 406 |
| 91 | nickel ferrite | 0.154 | Zn_{0.4}Ni_{0.6}Fe₂O₄ | 12 | TMAOH | | 406 |
| 92 | nickel ferrite | 0.154 | Zn_{0.4}Ni_{0.6}Fe₂O₄ | 12 | BSA | | 406 |
| 93 | nickel ferrite | 0.154 | Zn_{0.4}Ni_{0.6}Fe₂O₄ | 12 | carbodextran | | 406 |
| 94 | manganese | 0.154 | Zn_{0.4}Mn_{2.6}O₄ | 6 | TMAOH | | — |
| | oxide | | | | | | |
| 95 | manganese | 0.154 | Zn_{0.4}Mn_{2.6}O₄ | 6 | BSA | | — |
| | oxide | | | | | | |
| 96 | manganese | 0.154 | Zn_{0.4}Mn_{2.6}O₄ | 6 | carbodextran | | — |
| | oxide | | | | | | |
| 97 | cobalt oxide | 0.25 | Zn_{0.2}Co_{0.8}O | 7 | TMAOH | | — |
| 98 | cobalt oxide | 0.25 | Zn_{0.2}Co_{0.8}O | 7 | BSA | | — |
| 99 | cobalt oxide | 0.25 | Zn_{0.2}Co_{0.8}O | 7 | carbodextran | | — |
| 100 | nickel oxide | 0.25 | Zn_{0.2}Ni_{0.8}O | 10 | TMAOH | | — |
| 101 | nickel oxide | 0.25 | Zn_{0.2}Ni_{0.8}O | 10 | BSA | | — |
| 102 | nickel oxide | 0.25 | Zn_{0.2}Ni_{0.8}O | 10 | carbodextran | | — |

(*DMSA: dimercaptosuccinic acid, TMAOH: tetramethylammoniumhydroxide pentahydrate)

According to a preferable embodiment, the nanoparticle of the present invention has a specific loss power value in a range of 2-20000 W/g, more preferably 50-10000 W/g, much more preferably 100-5000 W/g and most preferably 200-5000 W/g.

In another view, the present invention provide the zinc-containing metal oxide-biologically/chemically active hybrid nanoparticles in which chemically functional molecules or biological materials having the biofunctional property are combined with the reactive region of the zinc-containing metal oxide nanomaterials.

As used in this invention, the “hybrid nanoparticles of zinc-containing metal oxide nanomaterials and biologically/chemically active materials” refers to nanoparticles in which bioactive materials (example: an antibody, a protein, an antigen, a peptide, a nucleic acid, an enzyme, a cell, etc.) or chemically active materials (example: a monomer, a polymer, an inorganic material, a fluorescent material, a drug, etc.) are bound to the active ingredient of the ligand in the zinc-containing metal oxide nanomaterials through covalent bond, ionic bond or hydrophobic interaction.

Further examples of the biomolecules include, but not limited to, a protein, a peptide, a nucleic acid, an antigen, an enzyme, a cell and a biofunctional drug, and preferably tissue-specific substances such as an antigen, an antibody, DNA, RNA, hapten, avidin, streptavidin, neutravidin, protein A, protein G, lectin and selectin; pharmaceutical active ingredients such as an anti-cancer drug, an antibiotic, a hormone, a hormone antagonist, interleukin, interferon, a growth factor, a tumor necrosis factor, endotoxin, lymphotoxin, urokinase, streptokinase, a tissue plasminogen activator, a protease inhibitor, alkyl phosphocholine, a surfactant, pharmaceutically active ingredients such as cardiovascular pharmaceuticals, gastrointestinal pharmaceuticals and neuro pharmaceuticals; biological active enzymes such as hydrolase, oxido-reductase, lyase, isomerase and synthetase; an enzyme cofactor; and an enzyme inhibitor.

The chemically active materials include several functional monomers, polymers, inorganic materials, fluorescent organic materials or drugs.

Exemplified monomer described herein above includes, but not limited to, a drug containing anti-cancer drugs, antibiotics, Vitamins and folic acid, a fatty acid, a steroid, a hormone, a purine, a pyrimidine, monosaccharides and a disaccharides. The side chain of the above-described monomer includes one or more functional groups selected from the group consisting of —COON, —NH₂, —SH, —SS—, —CONH₂, —PO₃H, —OPO₄H₂, —PO₂(OR¹)(OR²)(R¹, R²═CₛHₜNᵤO_{w}SₓP_{y}X_{z}, X═—F, —Cl, —Br or —I, 0≦s≦20, 0≦t≦2(s+u)+1, 0≦x≦2s, 0≦y≦2s, 0≦z≦2s), —SO₃H, —OSO₃H, —NO₂, —CHO, —COSH, —COX, —COOCO—, —CORCO—(R═CₗHₘ, 0≦l≦3, 0≦m≦21+1), —COOR, —CN, —N₃, —N₂, —NROH (R═CₛHₜNᵤO_{w}SₓP_{y}X_{z}, X═—F, —Cl, —Br or —I, 0≦s≦20, 0≦t≦2(s+u)+1, 0≦u≦2s, 0≦w≦2s, 0≦x≦2s, 0≦y≦2s, 0≦z≦2s), —NR¹NR²R³ (R¹, R², R³═CₛHₜNᵤO_{w}SₓX_{z}, X═—F, —Cl, —Br or —I, 0≦s≦20, 0≦t≦2(s+u)+1, 0≦u≦2s, 0≦w≦2s, 0≦x≦2s, 0≦y≦2s, 0≦z≦2s), —CONHNR¹R² (R¹, R²═CₛHₜNᵤO_{w}SₓP_{y}X_{z}, X=—F, —Cl, —Br or —I, 0≦s≦20, 0≦t≦2(s+u)+1, 0≦u≦2s, 0≦w≦2s, 0≦x2s, 0≦y≦2s, 0≦z≦2s), —NR¹R²R³X′ (R¹, R², R³═CₛHₜNᵤO_{w}SₓP_{y}X_{z}, X=—F, —Cl, —Br or —I, X′=F⁻, Cr⁻, Br⁻ or I⁻, 0≦s≦20, 0≦t≦2(s+u)+1, 0≦u≦2s, 0≦w≦2s, 0≦x≦2s, 0≦y≦2s , 0≦z≦2s), —OH, —SCOCH₃, —F, —Cl, —Br, —I, —SCN, —NCO, —OCN, -epoxy group, -hydrazone group, -alkene group and alkyne group.

The example of the above-described chemical polymer includes dextran, carbodextran, polysaccharide, cyclodextran, pullulan, cellulose, starch, glycogen, monosaccharides, disaccharides and oligosaccharides, polyphosphagen, polylactide, polylactide-co-glycolide, polycaprolactone, polyanhydride, polymaleic acid and a derivative of polymaleic acid, polyalkylcyanoacrylate, polyhydroxybutylate, polycarbonate, polyorthoester, polyethylene glycol, poly-L-lysine, polyglycolide, polymethyl methacrylate, polymethylether methacrylate and polyvinylpyrrolidone, but not limited to.

Exemplified chemical inorganic material described above includes a metal oxide, a metal chalcogen compound, an inorganic ceramic material, a carbon material, a semiconductor substrate consisting of Group II/VI elements, Group III/VI elements and Group IV elements, a metal substrate or complex thereof, and preferably, SiO₂, TiO₂, ITO, nanotube, graphite, fullerene, CdS, CdSe, CdTe, ZnO, ZnS, ZnSe, ZnTe, Si, GaAs, AlAs, Au, Pt, Ag or Cu.

The example of the above-described chemical fluorescent material includes a fluorescent organic substance such as fluorescein and its derivatives, rhodamine and its derivatives, lucifer yellow, B-phytoerythrin, 9-acrydine isothiocyanate, lucifer yellow VS, 4-acetamido-4′-isothio-cyanatostilbene-2,2′-disulfonate, 7-diethylamino-3-(4′-isothiocyatophenyl)-4-methylcoumarin, succinimidyl-pyrenebutyrate, 4-acetoamido-4′-isothio-cyanatostilbene-2,2′-disulfonate derivatives, LC™-Red 640, LC™-Red 705, Cy3, Cy5, Cy5.5, Alexa dye series, resamine, isothiocyanate, diethyltriamine pentaacetate, 1-dimethylaminonaphthyl-5-sulfonate, 1-anilino-8-naphthalene, 2-p-toluidinyl-6-naphthalene, 3-phenyl-7-isocyanatocoumarin, 9-isothiocyanatoacridine, acridine orange, N-(p-(2-benzoxazolyl)phenyl)meleimide, benzoxadiazol, stilbene and pyrene, but not limited to.

Since the nanomaterials of the present invention exhibit superior heat-generation, it may be used not only in a variety of heat-generating devices but also in hyperthermia or drug release for biomedical purpose. In more detail, the heat-generating nanomaterials of the present invention may be applied to uses such as cancer treatment, pain relief, vessel treatment, bone recovery, drug activation or drug release.

In another aspect of this invention, there is provided a heat-generating composition comprising the zinc-containing magnetic nanomaterial represented by the following formulae 3-4, 6 or 9-10:

Zn_{f}M_{a-f}O_{b} Formula 3

(0<f<8, 0<a≦16, 0<b≦8, 0<f/(a-f)<10; M represents the magnetic metal atom or the alloy thereof); or

Zn_{g}M_{c-g}M′_{d}Oₑ Formula 4

(0<g<8, 0<c≦16, 0<d≦16, 0<e≦8, 0<g/{(c-g)+d}<10; M represents the magnetic metal atom or the alloy thereof; M′ represents an element selected from the group consisting of Group 1 metal elements, Group 2 metal elements, Group 12 elements, Group 13 elements, Group 14 elements, Group 15 elements, transition metal elements, Lanthanide metal elements and Actinide metal elements.

According to a preferable embodiment, the zinc-containing magnetic nanomaterial is represented by the following formula 6:

ZnₖM″ₕ₋ₖFeᵢOⱼ Formula 6

(0<k<8, 0<h≦16, 0<j≦8, 0<k/{(h-k)+i}<10; M″ represents the magnetic metal atom or the alloy thereof).

More preferably, the zinc-containing magnetic nanomaterial is represented by the following formula 9 or 10:

Zn_{q}Fe_{i-q}Oₘ Formula 9

(0<q<8, 0<l≦8, 0<m≦8, 0<q/(l-q)<10); or

ZnᵣMnₙ₋ᵣFeₒOₚ Formula 10

(0<r<8, 0<n≦8, 0<o≦8, 0<p≦8, 0<r/{(n-r)+o}<10).

According to a preferable embodiment, the nanoparticle of the present invention have a specific loss power value in a range of 2-20000 W/g, more preferably 100-5000 W/g, much more preferably 200-5000 W/g and most preferably 400-2000 W/g.

In still another aspect of this invention, there is provided a composition for hyperthermia comprising the present heat-generating composition as described above.

In still another aspect of this invention, there is provided a method for hyperthermia, which comprises administering to a subject the present heat-generating composition as described above.

Since the present composition comprises the nanoparticle for hyperthermia as active ingredients described above, the common descriptions between them are omitted in order to avoid undue redundancy leading to the complexity of this specification.

The composition of this invention may be provided as a pharmaceutical composition. Therefore, the composition of the present invention may be administrated together with a pharmaceutically acceptable carrier, which is commonly used in pharmaceutical formulations, but is not limited to, includes lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methylcellulose, methylhydroxy benzoate, propylhydroxy benzoate, talc, magnesium stearate and mineral oils. Details of suitable pharmaceutically acceptable carriers and formulations can be found in Remington's Pharmaceutical Sciences (19th ed., 1995), which is incorporated herein by reference.

The composition according to the present invention may be parenterally administered. In the case that the contrast agent is administered parenterally, it is preferably administered by intravenous, subcutaneous, intramuscular, intraperitoneal or intralesional injection. A suitable dosage amount of the composition of the present invention may vary depending on pharmaceutical formulation methods, administration methods, the patient's age, body weight, sex, pathogenic state, diet, administration time, administration route, an excretion rate and sensitivity for a used nanomaterial. The composition of the present invention includes a therapeutically effective amount of the heat-generating composition. The term “therapeutically effective amount” refers to an amount enough to show and accomplish images of human body and is generally administered with a daily dosage of 0.0001-100 mg/kg.

According to the conventional techniques known to those skilled in the ah, the pharmaceutical composition of the present invention may be formulated with pharmaceutically acceptable carrier and/or vehicle as described above, finally providing several forms including a unit dose form and a multi-dose form. Non-limiting examples of the formulations include, but not limited to, a solution, a suspension or an emulsion in oil or aqueous medium, an elixir, a powder, a granule, a tablet and a capsule, and may further comprise a dispersion agent or a stabilizer.

In particular, the composition of the present invention is very useful in cancer treatment. For example, the present composition may effectively induce cancer cell apoptosis in various cancer diseases such as stomach, lung, breast, ovarian, liver, bronchogenic, nasopharyngeal, laryngeal, pancreatic, bladder, colon, cervical, brain, prostatic, bone, skin, thymus, hyperthymus and ureteral carcinoma.

The nanoparticle may be used in a form combined with a targeting molecule which is specifically bound to a target cell or not. The targeting molecule permits core-shell type nanoparticles to kill the target cells using hyperthermia by specific binding to cells of interest. The targeting molecule which is able to be used in the present invention includes, but not limited to, an antibody (preferably, a monoclonal antibody) against a surface antigen of a target cell, an aptamer, a receptor, lectin, DNA, RNA, a ligand, an coenzyme, an inorganic ion, an enzyme cofactor, a saccharide, a lipid and a substrate.

The composition of the present invention is administrated into a patient through suitable administration route and then is kept to stand under magnetic field of high frequency, resulting in heat generation. High frequency magnetic field of electromagnetic wave having the frequency of from 1 kHz to 10 MHz may be utilized.

The features and advantages of the present invention will be summarized as follows:

(i) The present invention suggests a novel approach to improve the heat generation of magnetic nanomaterials.

(ii) According to the present invention, the heat generation can be controlled by changing a zinc-content to be contained in nanomaterials.

(iii) According to the present invention, a composition for hyperthermia showing controlled specific loss power can be successfully provided.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1

### Preparation of Zinc-Containing Metal Oxide Nanomaterials

The metal oxide nanomaterial used in Examples was produced according to the methods described in Korean Pat. No. 10-0604975 PCT/KR2004/003088 and Korean Pat. No. 2006-0018921 filed by the present inventors. As precursors of nanoparticles, ZnCl₂ (Aldrich, USA), MCl₂ (M=Mn²⁺, Fe²⁺, Ni²⁺, and Co²⁺) (Aldrich, USA) and Fe(acac)₃ (Aldrich, USA) were added to trioctylamine solvent (Aldrich, USA) containing 20 mmol oleic acid (Aldrich, USA) and 20 mmol oleylamine (Aldrich, USA) as capping molecules. The mixture was incubated at 200° C. under argon gas atmosphere and further reacted at 300° C. The synthesized nanoparticles were precipitated by excess ethanol and then the precipitated nanoparticles were again dispersed in toluene, obtaining a colloid solution. The synthesized nanoparticles were 15 nm-sized Zn_{0.4}M_{0.6}Fe₂O₄ (M=Mn²⁺, Fe²⁺, Ni²⁺, and Co²⁺) nanoparticles.

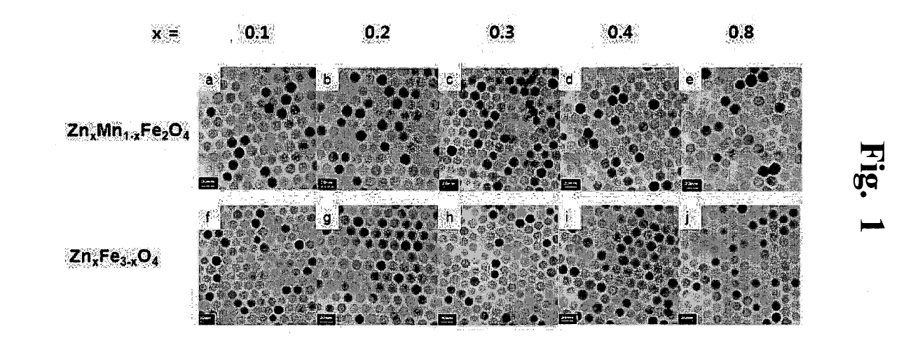

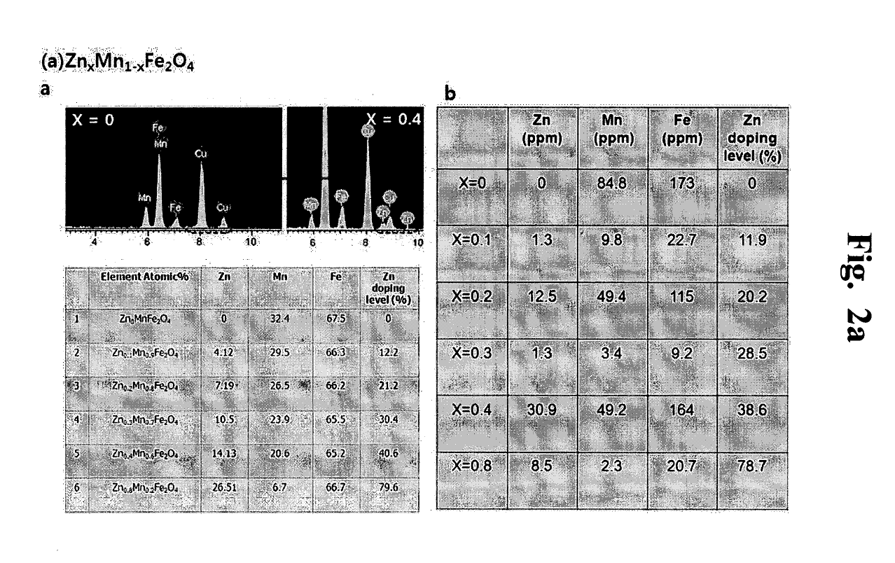

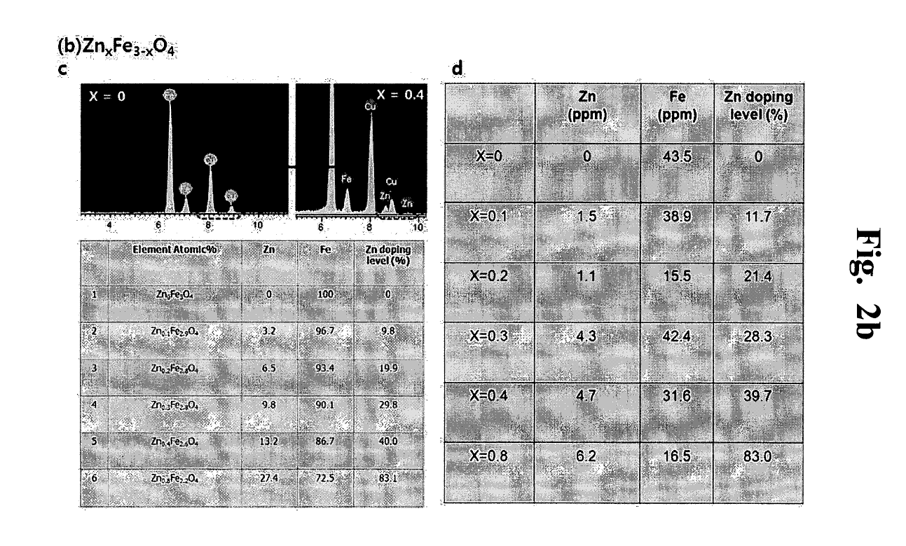

In addition, composition could be feasibly varied depending on the relative mole number of MCl₂ (M=Mn²⁺, Fe²⁺, Ni²⁺, Co²⁺, and Zn²⁺) materials as initial reactants. The synthetic nanoparticles have globular structure with a homogeneous size and their characteristics were analyzed using TEM (transmission electron microscopy), EDAX (Energy dispersive atomic emission spectra of X-ray) and ICP-AES (Inductively coupled plasma atomic emission spectra). TEM images of nanoparticles produced were shown in FIG. 1, and DEAX and ICP of nanoparticles produced were shown in FIG. 1 and FIG. 2.

### Example 2

### Comparison of Saturation Magnetization (Mₛ) of ZnₓM₁₋ₓFe₂O₄ (M=Fe or Mn, x=0, 0.1, 0.2, 0.3, 0.4, and 0.8) with Varying Zinc Content

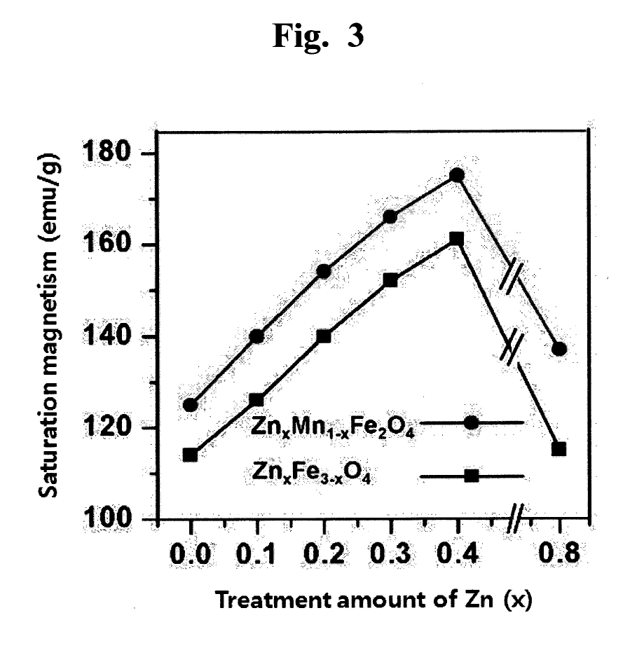

15 nm-sized ZnₓM₁₋ₓFe₂O₄ (M=Fe or Mn, x=0, 0.1, 0.2, 0.3, 0.4, and 0.8) nanoparticles were synthesized according to the method in Example 1 and their saturation magnetizations (Mₛ) in 3 Tesla were measured using a SQUID (Superconducting Quantum Interference Devices). As a result, each saturation magnetization (Mₛ) of ZnₓM₁₋ₓFe₂O₄ (M=Fe or Mn, x=0, 0.1, 0.2, 0.3, 0.4, and 0.8) nanoparticles was 125, 140, 154, 166, 175 and 137 emu/g (Zn+Mn+Fe), respectively. Likewise, each saturation magnetization (Mₛ) of ZnₓFe₃₋ₓO₄ (x=0, 0.1, 0.2, 0.3, 0.4, and 0.8) nanoparticles also was 114, 126, 140, 152, 161 and 115 emu/g (Zn⁺ Fe), respectively. The saturation magnetization (Mₛ) of synthesized nanoparticles was represented in FIG. 3.

### Example 3

### Comparison of Heat Generation of ZnₓM₁₋ₓFe₂O₄ (M=Fe or Mn, x=0, 0.1, 0.2, 0.4, and 0.8) with Varying Zinc Content

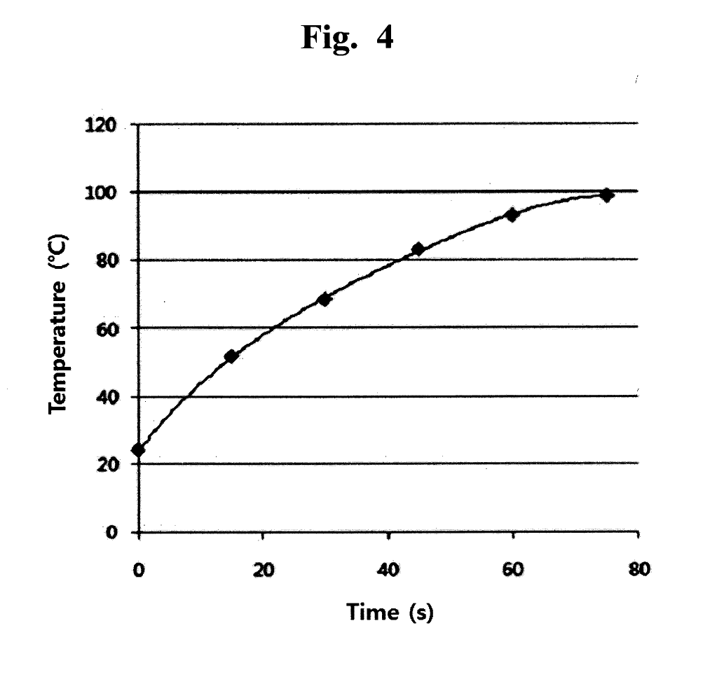

To systematically compare heat generation value of ZnₓM₁₋ₓFe₂O₄ (M=Fe or Mn, x=0, 0.1, 0.2, 0.4, and 0.8), heat generation from ZnₓM₁₋ₓFe₂O₄ (M=Fe or Mn, x=0, 0.1, 0.2, 0.4, and 0.8) nanoparticles with different zinc amount under the magnetic field of high frequency was measured under condition of the equal concentration. Based on the time-dependent temperature changes in coil with 5 cm diameter in 5 mg/mL solution under the alternative current magnetic field (frequency: 500 kHz, current: 35 A) (FIG. 4), the specific loss powers of the nanoparticles were measured.

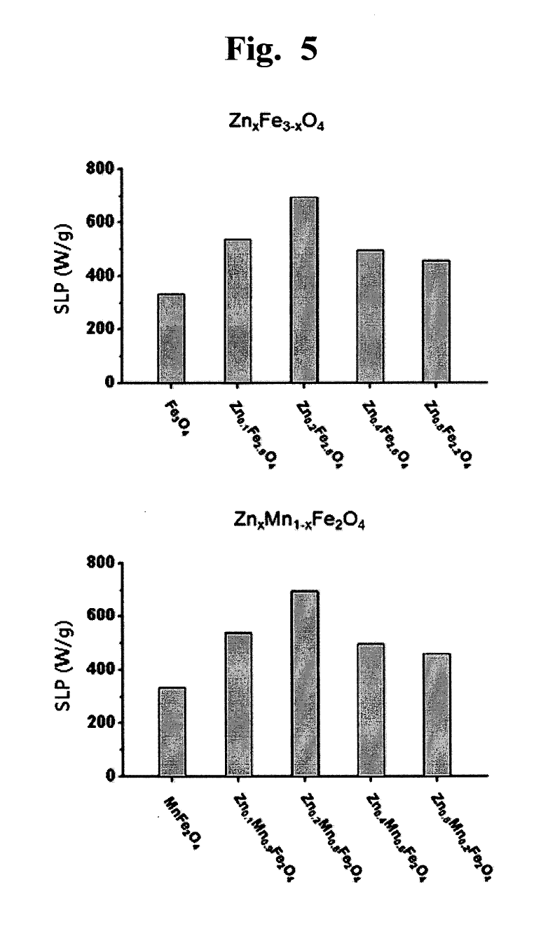

In the case adding zinc to the metal oxide nanomaterial such as manganese ferrite or iron oxide, the heat generation coefficient value is varied depending on the addition of zinc-content. Briefly, it is as follows. It was observed that the heat generation of both 15 nm-sized ZnₓM₁₋ₓFe₂O₄ and ZnₓFe₃₋ₓO₄ nanoparticles is controlled according to change of zinc-content. In ZnₓFe₃₋ₓO₄ nanoparticles, specific loss power value (333, 539, 694, 496, and 458 W/g) was controlled according to the increase in zinc-content (x=0, 0.1, 0.2, 0.4, and 0.8), and in ZnₓM₁₋ₓFe₂O₄ nanoparticles, specific loss power value (411, 451, 667, 472, and 379 W/g) was controlled according to the increase in zinc-content (x=0, 0.1, 0.2, 0.4, and 0.8). These results are closely related to those analyzed in Example 2 in which the saturation magnetism is varied by the addition of zinc, and also suggest that the anisotropy constant of nanoparticles varied by the addition of zinc is responsible for their specific loss power as well as magnetic moments. Comparative data of heat generation of ZnₓM₁₋ₓFe₂O₄ (M=Fe²⁺or Mn²⁺, x=0, 0.1, 0.2, 0.4, and 0.8) according to the addition of zinc were represented in FIG. 5.

### Example 4

### Comparison of Heat Generation of Various Zinc-Doped Metal Oxides

To investigate whether specific loss power is changed by adding zinc to various metal oxides, MFe₂O₄ (M=Mn²⁺, Fe²⁺, Co²⁺, and Ni²⁺), the nanoparticles with equal size (15 nm) were synthesized and their specific loss powers were measured. The zinc-containing iron oxide nanoparticles were synthesized according to the methods described in Korean Pat. No. 10-0604975, No. 10-0652251, No. 10-0713745, PCT/KR2004/002509, Korean Pat. No. 10-0604975, PCT/KR2004/003088, PCT/KR2007/001001 and Korean Pat. Appln. No. 2006-0018921. As a result, it is demonstrated that all four nanoparticles has enhanced heat generation in the case (Zn_{0.2}M_{0.8}Fe₂O₄, x=0.2) adding zinc to various metal oxides, MFe₂O₄ (M=Mn²⁺, Fe²⁺, Co²⁺, and Ni²⁺).

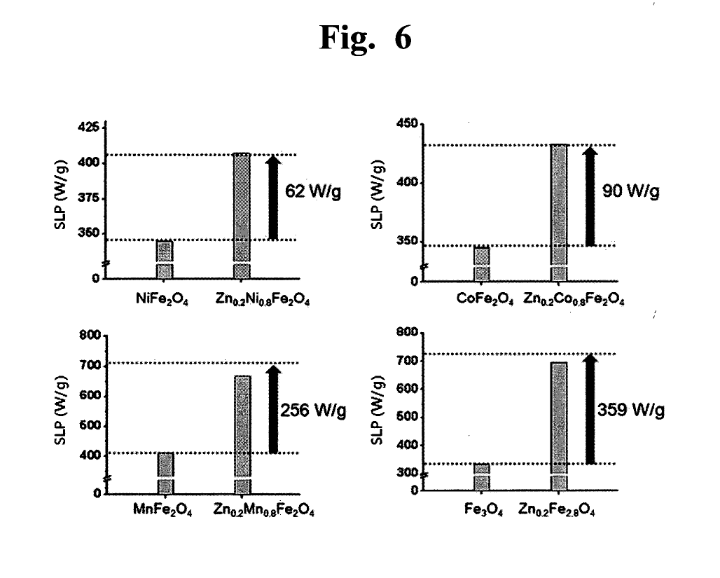

Comparative data of heat generation of Zn_{0.2}M_{0.8}Fe₂O₄ (M=Mn²⁺, Fe²⁺, Co²⁺, and Ni²⁺) according to the addition of zinc were represented in FIG. 6.

### Example 5

### Synthesis of Zinc-Containing Ferrite Nanomaterials with the Composition of ZnₓFe₃₋ₓO₄ (x=0.2, 0.4) in Aqueous Solution

The heat-generating agent containing the zinc-containing metal oxide nanomaterial suggested by the present invention may not be limited to the nanoparticles synthesized through a phase transfer process in the above-described organic solvent but be produced by the following method in an aqueous solution. To synthesize the zinc-containing metal oxide nanomaterials having the composition of ZnₓFe₃₋ₓO₄ (x=0.2, 0.4) solubilized in water, Zn(acac)₂H₂O 20 mg, FeCl₂H₂O 60 mg and FeCl₃H₂O 240 mg were dissolved in 10 mL H₂O, and were added to 1 mL of 3.2 M NH₄OH solution, followed by reacting for 20 min with vigorous shaking to obtain Zn_{0.2}Fe_{0.8}Fe₂eO₄ nanoparticles. In addition, each Zn(acac)₂H₂O and FeCl₂H₂O as precursor of nanoparticles was used in an amount of 40 mg and then reacted under the same conditions, obtaining Zn_{0.4}Fe_{2.6}O₄ nanoparticles.

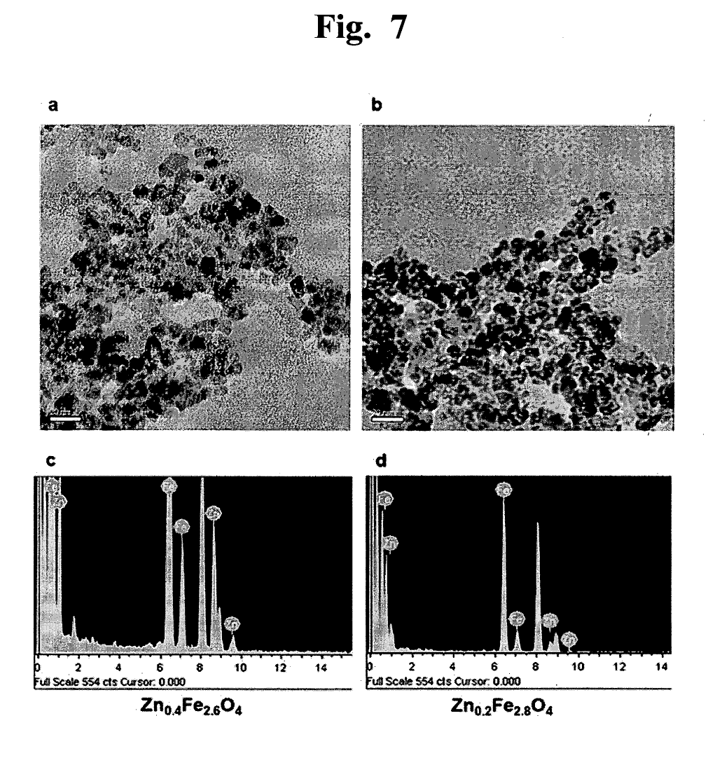

FIG. 7 represents an analysis of zinc-content using TEM (FIGS. 7a-7b) or EDAX (FIGS. 7c-7d) of ZnₓFe₃₋ₓO₄ (x=0.2, 0.4) nanoparticles synthesized according to the above-described method.

### Example 6

### Synthesis of Zinc-Containing Ferrite Nanomaterials with the Composition of ZnₓM₁₋ₓFe₂O₄ (M=Mn²⁺, Fe²⁺, Co²⁺, Ni²⁺, x=0, 0.1, 0.2, 0.3, 0.4, and 0.8) and the Core Size of 15 nm Coated with Dimercaptosuccinic Acid (DMSA)

The nanoparticles dispersed in 20 mg/mL toluene solution were added to DMSO solution containing excess dimercaptosuccinic acid (DMSA) and reacted for 2 hrs. Thus, the nanoparticles were isolated using a centrifuge and then dispersed in water.

### Example 7

### Synthesis of Zinc-Containing Ferrite Nanomaterials with the Composition of ZnₓM₁₋ₓFe₂O₄ (M=Mn²⁺, Fe²⁺, Co²⁺, Ni²⁺, x=0, 0.1, 0.2, 0.3, 0.4, and 0.8) and the Core Size of 6, 9, 12 nm Coated with Dimercaptosuccinic Acid (DMSA)

The nanoparticles dispersed in 20 mg/mL toluene solution were added to DMSO solution containing excess dimercaptosuccinic acid (DMSA) and reacted for 2 hrs. Thus, the nanoparticles were isolated using a centrifuge and then dispersed in water.

### Example 8

### Synthesis of Zinc-Containing Oxide Nanomaterials with the Composition of ZnₓM₁₋ₓFe₂O₄=Mn²⁺, Fe²⁺, Co²⁺, and Ni²⁺, x=0, 0.1, 0.2, 0.3, 0.4, and 0.8) Coated with Tetramethylammoniumhydroxide Pentahydrate (TMAOH) as a Heat-Generating Agent

The zinc-containing oxide nanoparticles (50 mg/mL) dispersed in 1 mL toluene solution were precipitated using excess ethanol and re-dispersed in 5 mL TMAOH (tetramethylammoniumhydroxide pentahydrate) solution to disperse in aqueous solution.

### Example 9

### Evaluation on Capacity of Inducing Cancer Cell Apoptosis

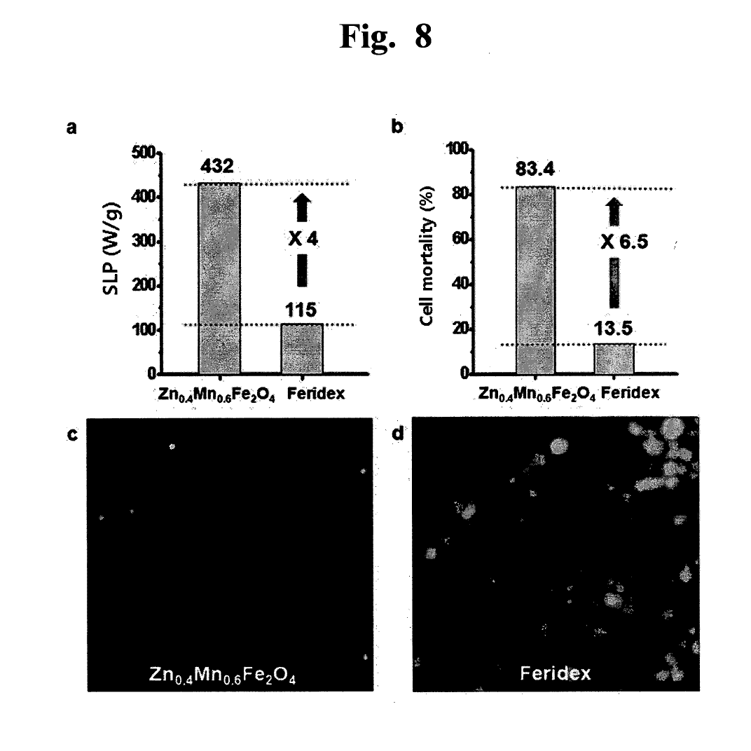

The nanomaterials having enhanced specific loss power may be applied in various fields. As a representative example, the nanomaterial is very efficiently used in cancer cell apoptosis. Based on the fact that cancer cells are likely to be killed around 40-50° C. unlikely normal cells, nanomaterials having enhanced heat-generation coefficient become promising as cancer hyperthermia agents by inducing cancer cell apoptosis because even a very low dose of nanomaterials generates higher heat. To verify the hyperthermic effect, the equal amounts of commercially purchasable Feridex and 15 nm-sized Zn_{0.4}Mn_{0.6}Fe₂O₄ nanoparticles of the present invention having remarked heat-generation coefficient were incubated with cancer cells (HeLa cells) and then kept to stand under magnetic field of high frequency. As a result, 15 nm-sized Zn_{0.4}Mn_{0.6}Fe₂O₄ nanoparticles induced much more considerable cancer cell apoptosis than conventional nanoparticles (see FIG. 8).

Having described a preferred embodiment of the present invention, it is to be understood that variants and modifications thereof falling within the spirit of the invention may become apparent to those skilled in this art, and the scope of this invention is to be determined by appended claims and their equivalents.

1. A method for controlling heat generation of a magnetic nanomaterial, comprising the steps of: (a) mixing (i) a nanomaterial precursor comprising a metal precursor material and a predetermined amount of a zinc precursor with (ii) a reaction solvent; and (b) preparing a zinc-containing magnetic nanomaterial from the mixture of step (a) comprising a zinc doped metal oxide nanomaterial matrix; and wherein a specific loss power of the zinc-containing magnetic nanomaterial is varied depending an amount of zinc to be doped, whereby the heat generation of the magnetic nanomaterial is controlled.

2. The method according to claim 1, wherein the zinc-containing magnetic nanomaterial comprises the metal oxide nanomaterial matrix in which a zinc atom is added to the metal oxide nanomaterial matrix to substitute a metal atom or to be added to a vacant interstitial hole

3. The method according to claim 1, wherein the zinc-containing magnetic nanomaterial comprises the metal oxide nanomaterial matrix represented by the following formula 1 or 2, in which the zinc atom is added to the metal oxide nanomaterial matrix to substitute a metal atom or to be added to a vacant interstitial hole, or is represented by the following formula 3 or 4:
MₐO_{b} Formula 1
(0<a<16, 0<b≦8, M represents a magnetic metal atom or the alloy thereof);
M_{c}M′_{d}Oₑ Formula 2
(0<c≦16, 0<d≦16, 0<e≦8; M represents the magnetic metal atom or the alloy thereof; M′ represents an element selected from the group consisting of Group 1 metal elements, Group 2 metal elements, Group 12 elements, Group 13 elements, Group 14 elements, Group 15 elements, transition metal elements, Lanthanide metal elements and Actinide metal elements);
Zn_{f}M_{a-f}O_{b} Formula 3
(0<f<8, 0<a≦16, 0<b≦8, 0<f/(a-f)<10; M represents the magnetic metal atom or the alloy thereof); or
Zn_{g}M_{c-g}M′_{d}Oₑ Formula 4
(0<g<8, 0<c≦16, 0<d≦16, 0<e≦8, 0<g/{(c-g)+d}<10; M represents the magnetic metal atom or the alloy thereof; M′ represents an element selected from the group consisting of Group 1 metal elements, Group 2 metal elements, Group 12 elements, Group 13 elements, Group 14 elements, Group 15 elements, transition metal elements, Lanthanide metal elements and Actinide metal elements).

4. The method according to claim 3, wherein M′ represents the element selected from the group consisting of Li, Na, K, Rb, Cs, Be, Mg, Ca, Sr, Ba, Ra, Ge, Ga, Bi, In, Si, Ge, Ti, V, Cr, Mn, Fe, Co, Ni, Cu, Y, Zr, Nb, Mo, Tc, Ru, Rh, Pd, Ag, Cd, Hf, Ta, W, Re, Os, Ir, Pt, Au, Hg, Lanthanide metal elements and Actinide metal elements.

5. The method according to claim 3, wherein the zinc-containing magnetic nanomaterial comprises the metal oxide nanomaterial matrix represented by the following formula 5, in which the zinc atom is added to the metal oxide nanomaterial matrix to substitute a metal atom or to be added to a vacant interstitial hole, or is represented by the following formula 6:
M″ₕFeᵢOⱼ Formula 5
(0<h≦16, 0<i≦8, 0<j<8; M″ represents the magnetic metal atom or the alloy thereof);
ZnₖM″ₕ₋ₖFeᵢOⱼ Formula 6
(0<k<8, 0<h≦16, 0<I 8, 0<j≦8, 0<k/{(h-k)+i}<10; M″ represents the magnetic metal atom or the alloy thereof).

6. The method according to claim 5, wherein the zinc-containing magnetic nanomaterial comprises the metal oxide nanomaterial matrix represented by the following formula 7 or 8, in which a zinc atom is added to the metal oxide nanomaterial matrix to substitute a metal atom or to be added to a vacant interstitial hole, or is represented by the following formula 9 or 10:
FeₗOₘ(0<l≦8, 0<m≦8) Formula 7
MnₙFeₒOₚ(0<n≦8, 0<o≦8, 0<p≦8) Formula 8
Zn_{g}Fe_{l-q}Oₘ(0<q<8, 0<l≦8, 0<m≦8, 0<q/(l-q)<10) Formula 9
ZnᵣMnₙ₋ᵣFeₒOₚ(0<r<8, 0<n≦8, 0<o≦8, 0<p≦8, 0<r/{(n-r)+o}<10). Formula 10

7. The method according to claim 1, wherein a stoichiometric ratio of zinc and other metal in the zinc-containing magnetic nanomaterial is in a range of 0.001<zinc/(entire metal material-zinc)<10.

8. The method according to claim 1, wherein the zinc-containing magnetic nanomaterial has the heat-generation coefficient higher than metal oxide nanomaterial matrix.

9. The method according to claim 1, wherein the reaction solvent of the step (a) is an organic solvent or an aqueous solution and the step (b) is carried out by decomposing the nanoparticle precursor in the reaction solvent to prepare the zinc-containing magnetic nanomaterial.

10. The method according to claim 1, wherein the nanoparticle precursor is selected from the group consisting of a metal nitrate-based compound, a metal sulfate-based compound, a metal acetylacetonate-based compound, a metal fluoroacetoacetate-based compound, a metal halide-based compound, a metal perchlorate-based compound, a metal alkyloxide-based compound, a metal sulfamate-based compound, a metal stearate-based compound and an organometallic compound.

11. The method according to claim 1, wherein the reaction solvent is selected from the group consisting of a benzene-based solvent, a hydrocarbon-based solvent, an ether-based solvent, a polymer-based solvent, an ionic liquid-based solvent, a halohydrocarbon-based solvent, an alcohol-based solvent and a sulfoxide-based solvent.

12-13. (canceled)

14. A heat-generating composition comprising the zinc-containing magnetic nanomaterial represented by the following formulae 3-4, 6 or 9-10:
Zn_{f}M_{a-f}O_{b} Formula 3
(0<f<8, 0<a≦16, 0<b≦8, 0<f/(a-f)<10; M represents the magnetic metal atom or the alloy thereof);
Zn_{g}M_{c-g}M′_{d}Oₑ Formula 4
(0<g<8, 0<c≦16, 0<d≦16, 0<e≦8, 0<g/{(c-g)+d}<10; M represents the magnetic metal atom or the alloy thereof; M′ represents an element selected from the group consisting of Group 1 metal elements, Group 2 metal elements, Group 12 elements, Group 13 elements, Group 14 elements, Group 15 elements, transition metal elements, Lanthanide metal elements and Actinide metal elements;
ZnₖM″ₕ₋ₖFeᵢOⱼ Formula 6
(0<k<8, 0<h≦16, 0<i≦8, 0<j≦8, 0<k/{(h-k)+i}<10; M″ represents the magnetic metal atom or the alloy thereof);
Zn_{q}Fe_{l-q}Oₘ Formula 9
(0<q<8, 0<l≦8, 0<m≦8, 0<q/(1-q)<10); or
ZnᵣMnₙ₋ᵣFeₒOₚ Formula 10
(0<r<8, 0<n≦8, 0<o≦8, 0<p≦8, 0<r/{(n-r)+o}<10).

15. The heat-generating composition according to claim 14, wherein the zinc-containing magnetic nanomaterial is represented by the following formula 6:
ZnₖM″ₕ₋ₖFeᵢOⱼ Formula 6
(0<k<8, 0<h≦16, 0<i≦8, 0<j≦8, 0<k/{(h-k)+i}<10; M″ represents the magnetic metal atom or the alloy thereof.

16. The heat-generating composition according to claim 14, wherein the zinc-containing magnetic nanomaterial is represented by the following formula 9 or 10:
Zn_{q}Fe_{l-q}Oₘ Formula 9
(0<q<8, 0<l≦8, 0<m≦8, 0<q/(1-q)<10); or
ZnᵣMnₙ₋ᵣFeₒOₚ Formula 10
(0<r<8, 0<n≦8, 0<o≦8, 0<p<8, 0<r/{(n-r)+o}<10).

17-18. (canceled)

19. A method for hyperthermia, which comprises administering to a subject the heat-generating composition according to claim 14.

20. A method for hyperthermia, which comprises administering to a subject the heat-generating composition according to claim 15.

21. A method for hyperthermia, which comprises administering to a subject the heat-generating composition according to claim 16.
